(19) United States
(12) Patent Application Publication
(10) Pub. No.: US 2020/0214828 A1
(43) Pub. Date: Jul. 9, 2020

(54) Bone Anchor and Related Devices, Systems, and Methods

(71) Applicant: Roberto Ivan Diaz, Los Gatos, CA (US)

(72) Inventor: Roberto Ivan Diaz, Los Gatos, CA (US)

(21) Appl. No.: 16/737,266

(22) Filed: Jan. 8, 2020

Related U.S. Application Data
(60) Provisional application No. 62/790,129, filed on Jan. 9, 2019.

(51) Int. Cl. A61F 2/08; A61B 17/04

(52) U.S. Cl. CPC A61F 2/0811; A61F 2002/30116; A61B 17/0401

(57) ABSTRACT

Disclosed herein are various attachment devices for anchoring tendons within bone and associated methods and systems. Disclosed is a device for repairing/reconstructing a ligament rupture via compression and grasping. The device includes first and second elongate body portions, where each elongate body portion includes at least one elongate member having a first end and a second end and a plurality of bands coupled to the at least one elongate member. The device further includes a connection structure disposed at a distal end of the at least one elongate member connecting the first and second elongate body portions such the first and second elongate body portions are movable in relation to each other. The device also includes an lumen defined by the first and second elongate body portions for insertion of a tendon graft.

### CROSS-REFERENCE TO RELATED APPLICATIONS

This application claims the benefit under 35 U.S.C. § 119(e) to U.S. Provisional Application 62/790,129, filed Jan. 9, 2019, and entitled “Bone Anchor and Related Devices, Systems, and Methods,” which is hereby incorporated herein by reference in its entirety for all purposes.'

### FIELD

The various embodiments disclosed herein relate to medical devices, especially medical devices for use in orthopedic surgery, and further to devices for anchoring tendon to bone.

### BACKGROUND

Ligament ruptures are common injuries that can cause pain, instability, and dysfunction in joints. Known ligament reconstruction procedures using a tendon graft are a common technique used in the treatment of ligament ruptures not amenable to primary repair (which involves direct repair of the patient's own torn ligament rather than use of a graft).

Currently, ligament reconstruction is typically done using bone anchors and/or biotenodesis screws. Biotenodesis screws use interference fixation to connect a tendon to bone. Using this known technique, the tendon is compressed between the screw and bone to achieve interference fixation of the tendon to the bone. The biotenodesis screw is typically of a larger diameter than the tendon being inserted into the bone. The larger the tendon being used in ligament reconstruction, the larger the screw and bone cavity required. One disadvantage of the biotenodesis screws is that the larger cavities required for the screws can create stress risers and fractures. A further disadvantage is the difficulty in creating the required large cavities in small bone. In situations in which the tendon is relatively large, yet another disadvantage is that insertion of the anchor and tendon graft into a bore can be challenging.

There is a need in the art for improved devices, systems, and methods for anchoring tendon to bone.

### BRIEF SUMMARY

Discussed herein are various devices, systems, and methods for anchoring tendon to bone.

In Example 1, an anchoring device for repairing a ligament comprises first and second elongate body portions, wherein each elongate body portion comprises at least one elongate member having a first end and a second end, and a plurality of bands coupled to the at least one elongate member. Further, the device comprises a connection structure disposed at a distal end of the at least one elongate member connecting the first and second elongate body portions, and a lumen defined by the first and second elongate body portions, wherein the first and second elongate body portions are movable in relation to each other via the connection structure.

Example 2 relates to the anchoring device for repairing a ligament according to Example 1, wherein each of the first and second elongate body portions has a semi-circular cross-section.

Example 3 relates to the anchoring device for repairing a ligament according to Example 1, wherein the lumen is configured to accept a tendon graft.

Example 4 relates to the anchoring device for repairing a ligament according to Example 3, wherein the device is shaped for insertion into a bore.

Example 5 relates to the anchoring device for repairing a ligament according to Example 4, wherein the first and second elongate body portions are configured to compress the tendon graft upon insertion into the bore.

Example 6 relates to the anchoring device for repairing a ligament according to Example 1, wherein each of the plurality of bands further comprise a first side and a second side.

Example 7 relates to the anchoring device for repairing a ligament according to Example 6, further comprising a plurality of barbs on the first side of each of the plurality of bands.

In Example 8, a method of repairing a ligament comprises attaching a suture to a first end of a tendon and providing an anchoring device. The anchoring device comprises first and second elongate body portions, each of the first and second elongate body portions comprising at least one elongate member having a proximal end and a distal end, and one or more bands coupled to the at least one elongate member. The anchoring device further comprises a connection disposed at a distal end of the at least one elongate member, the connection configured to allow movement of the first and second elongate body portion relative to each other, and a lumen defined by the first and second elongate body portions, the lumen having a first diameter. The method further comprises creating a bore in a bone, inserting the first end of the tendon into the lumen of the anchoring device, and inserting the anchoring device into the bore.

Example 9 relates to the method according to Example 8, further comprising urging the first and second elongate body portion toward each other such that the lumen is compressed to a second diameter, wherein the second diameter is smaller than the first diameter.

Example 10 relates to the method according to Example 9, further comprising urging the first and second elongate body portions away from each other such that the lumen is expanded to a third diameter, wherein the third diameter is larger than the first diameter.

Example 11 relates to the method according to Example 10, wherein the anchoring device further comprises a plurality of barbs disposed on the one or more bands and the plurality of barbs extending into the lumen.

Example 12 relates to the method according to Example 8, further comprising threading the suture through the lumen.

Example 13 relates to the method according to Example 8, further comprising tensioning the tendon.

In Example 14, an anchoring device comprises an elongate body comprising a first elongate body structure and a second elongate body structure disposed adjacent to the first elongate body structure. The anchoring device further comprises a lumen defined between the first and second elongate body structures, wherein the lumen has a substantially circular cross-section and a first diameter, and a distal connection structure disposed at a distal end of the elongate body, the distal connection structure coupled to the first and second central supports, wherein the first and second central supports are moveable in relation to each other via the distal connection structure. The first elongate body structure comprises a first central support, and a plurality of first ribs, wherein each of the plurality of first ribs is coupled to the first central support. The second elongate body structure comprises a second central support, and a plurality of second ribs, wherein each of the plurality of second ribs is coupled to the second central support.

Example 15 relates to the anchoring device according to Example 14, wherein the distal connection structure is a hinge-like structure, wherein the first and second central supports are moveable between an expanded position and a non-expanded position at the hinge-like structure.

Example 16 relates to the anchoring device according to Example 14, further comprising a plurality of protrusions extending outwardly from the first and second elongate body structures.

Example 17 relates to the anchoring device according to Example 14, wherein the plurality of first ribs and the plurality of second ribs are substantially triangular such that a point extends outwardly form the first and second elongate body structures.

Example 18 relates to the anchoring device according to Example 14, further comprising a plurality of barbs extending inwardly from the first and second elongate body structures.

Example 19 relates to the anchoring device according to Example 14, wherein the distal connection structure is an eyelet.

Example 20 relates to the anchoring device according to Example 14, wherein the distal connection structure is a bar.

Example 21 relates to the anchoring device according to Example 14, further comprising a plurality of protrusions extending outwardly from the first and second elongate body structures, and a plurality of barbs extending inwardly from the first and second elongate body structures.

Example 22 relates to the anchoring device according to Example 21, wherein at least two of the plurality of barbs extending inwardly are oriented distally and at least two of the plurality of barbs extending inwardly are oriented proximally.

Example 23 relates to the anchoring device according to Example 14, wherein the distal connection structure is sized and shaped to receive a suture or tendon such that the suture or tendon is disposed around or threaded through the distal connection structure.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a perspective view of the anchoring system with tendon graft, according to one embodiment.

FIGS. 2A and 2B are side perspective views of the anchoring device, according to one embodiment.

FIG. 3 is a side view of another anchoring device, according to another embodiment.

FIG. 4 is a side view of a further anchoring device, according to a further embodiment.

FIG. 5 is a side view of yet another anchoring device, according to another embodiment.

FIG. 6 is a side view of still another anchoring device, according to yet another embodiment.

FIG. 7 is a top view of an anchoring device, according to one embodiment.

FIG. 8 is a top view of another anchoring device, according to another embodiment.

FIG. 9 is a top view of a further anchoring device, according to a further embodiment

FIG. 10 is a bottom view of yet another anchoring device, according to another embodiment.

FIG. 11A is a side view of an anchoring device in an expanded position, according to one embodiment.

FIG. 11B is a side view of the anchoring device of FIG. 11A in a compressed position, according to one embodiment.

FIG. 12 is a cross-sectional view of a bone and bore with an anchoring device inserted, according to one embodiment.

FIG. 13 is a front view of the anchoring system implanted into two bones of a patient's wrist, according to one embodiment.

FIG. 14A is a side view of an anchoring device, according to one embodiment.

FIG. 14B is a perspective view of the anchoring device of FIG. 14A, according to one embodiment.

### DETAILED DESCRIPTION

The various embodiments disclosed herein relate to a device for anchoring a tendon to a bone, and, in some cases, a system of two such devices for anchoring both ends of a tendon to two different bones or bone areas. These implementations can be used to maximize the size of the tendon being implanted while minimizing the size of cavity needed in the bone. The anchoring device allows for tensioning of the tendon after insertion into the bone. The shape of the anchoring device can be adjusted as desired to create a narrower or wider tendon footprint, as needed. The device allows for anchoring tendon in bone without crushing the tendon as required by prior known devices and methods. Further, openings in the device allow for biological healing of the tendon with surrounding bone.

In certain specific implementations, the various anchoring device embodiments disclosed or contemplated herein may be used in connection with various known procedures, including, for example, scapholunate ligament reconstruction, thumb collateral ligament reconstruction, elbow collateral ligament reconstruction, distal biceps repair, proximal biceps repair, ACL reconstruction, knee collateral ligament reconstruction, ankle stabilization, tendon transfer, syndesmosis stabilization, spring/deltoid ligament repair, and other procedures as would be appreciated by those of skill in the art.

In various embodiments, the anchoring device implementations herein are made from any material that is flexible, strong, and manufacturable, as known and recognized by those of skill in the art. The various anchoring device embodiments may be made from metal, PEEK, or any other known bio-composite material, or any combination thereof, as would be known to those of skill in the art. The anchoring device implementations may be made in various sizes and shapes to best fit various patient anatomies and repairs. Further, the elongate anchoring device embodiments herein may be made in different cross-sectional shapes, such as a circular or oval-shaped cross-section, depending on the repair. For example, in certain circumstances, the oval-shaped cross-section may be more effective for repair of ligaments having a broad insertion, such as repair of the elbow lateral collateral ligament.

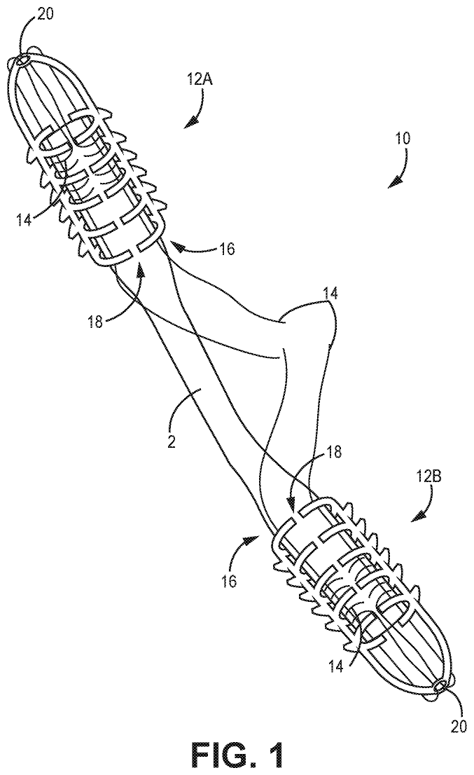

FIG. 1 depicts one embodiment of a system 10 using two anchoring devices 12A, 12B. According to one embodiment, a tendon graft 2 is inserted into devices 12A, 12B as shown. In some embodiments, in use, prior to insertion of the tendon 2 into each of the devices 12A, 12B, a suture 14 is placed along each end of the tendon 2 such that the suture 14 is attached to the tendon 2, in a manner known to those of skill in the art. After attachment of suture 14 to one end of the tendon 2, the suture 14 is inserted/threaded through the opening 16 and lumen 18 of the device 12A, 12B into which the tendon 2 is going to be inserted, and then threaded over or through a connection structure 20 of the device 12A, 12B, such as a connecting bar 20 or eyelet 20. After threading over or through the connection structure 20, the suture 14 may then be extended proximally back through the lumen 18 and back out of the opening 16 such that both ends of the suture 14 extend out of the device 12A, 12B as shown in FIG. 1. The suture 14 may then be used to draw or pull the tendon 2 into the devices 12A, 12B by pulling on the suture 14 ends. This process is then repeated for the second of the two anchoring devices 12A, 12B such that the tendon 2 is disposed within both devices 12A, 12B as shown.

Once the tendon 2 is attached to the anchoring devices 12A, 12B, the system 10 (both anchoring devices 12A, 12B with the inserted tendon 2) can then be inserted into the target bone for ligament reconstruction or repair. After insertion, further tension can then be applied to the tendon 2 (to urge the tendon 2 further into the lumen of either or both devices 12A, 12B) by additional pulling of the ends of the suture 14. By pulling the ends of the suture 14 associated with either device 12A, 12B, the tendon 2 is advanced further into the lumen 21 of that device 12A, 12B.

In some embodiments, once the tendon 2 is appropriately positioned within the devices 12A, 12B and bone and appropriate tension and length are achieved, the ends of the suture 14 may be tied together to augment the repair. In further embodiments, the ends of the suture 14 may also be trimmed, as necessary.

According to certain embodiments, the inner barbs 36, as described below, as well as the suture 14, hold the tendon 2 in place within the device 12A, 12B.

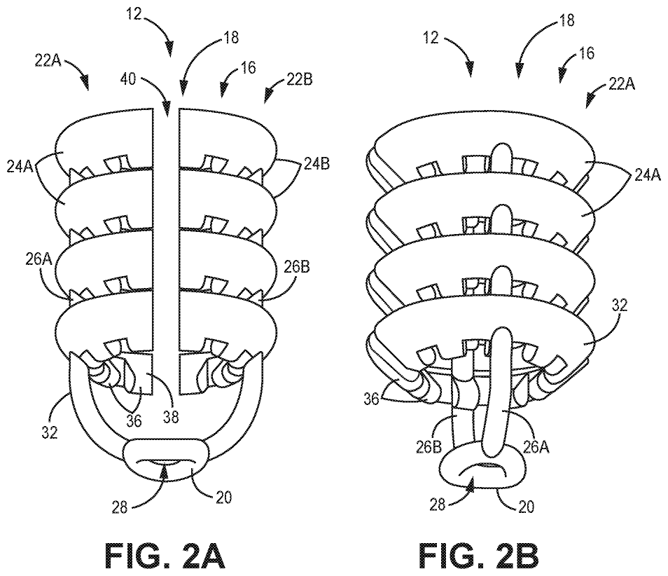

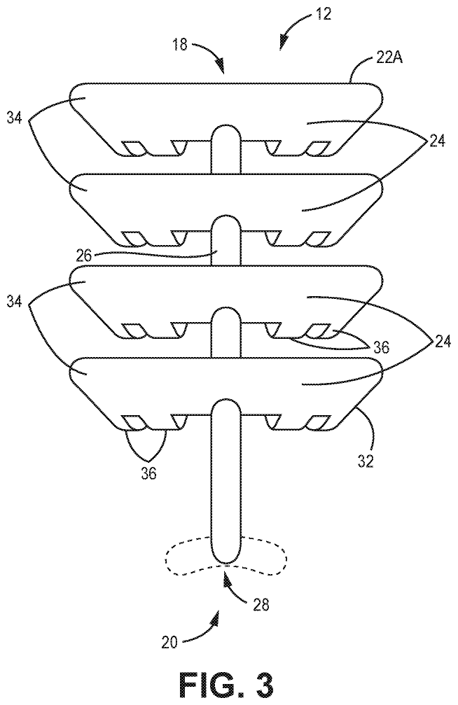

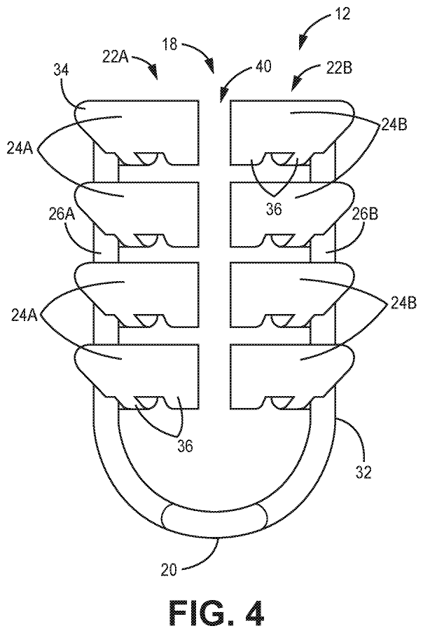

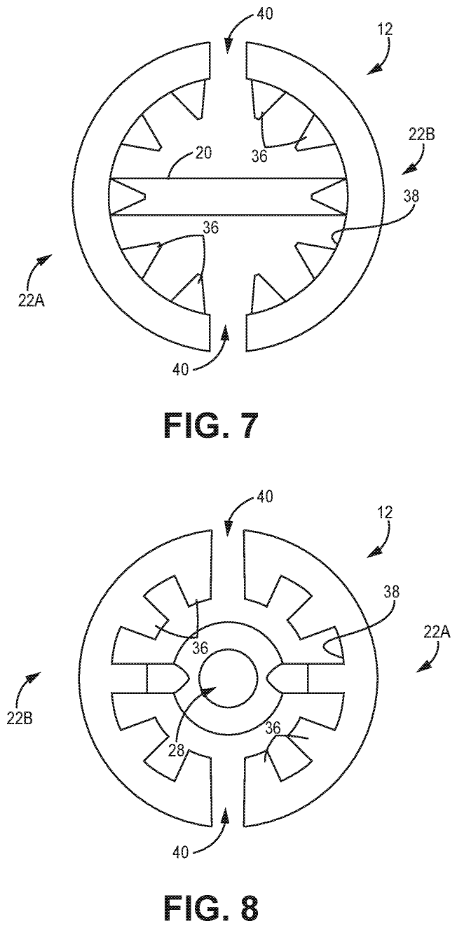

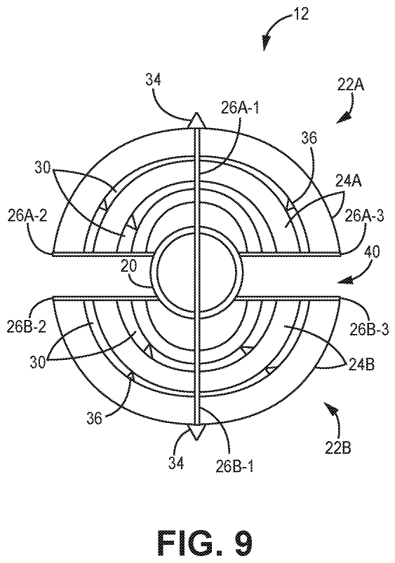

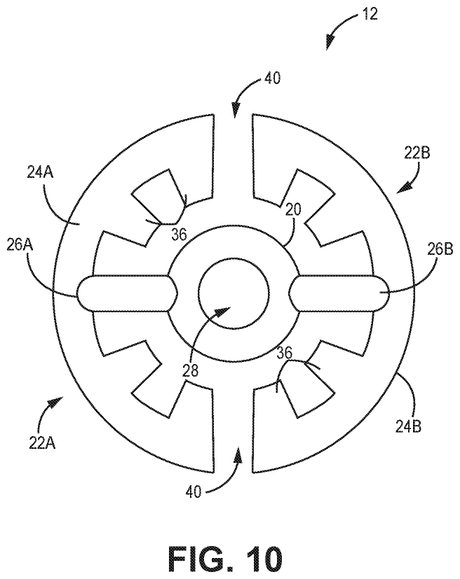

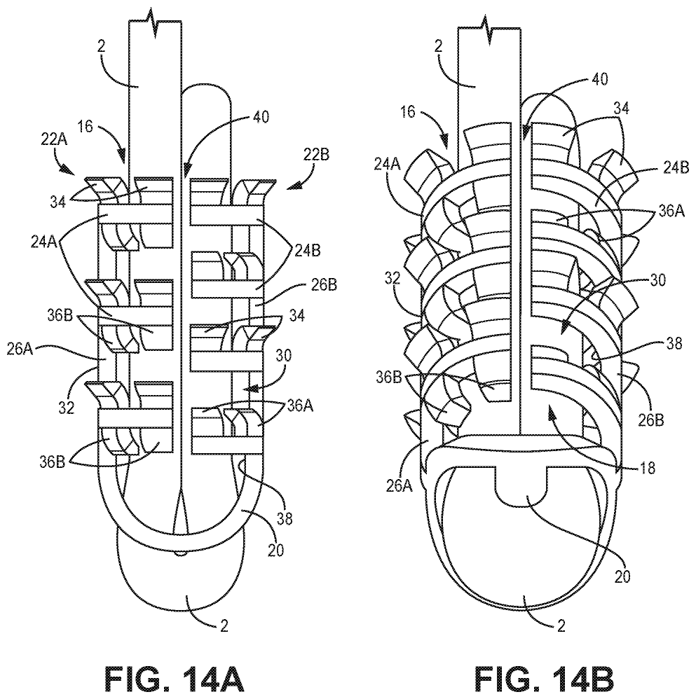

Turning to FIGS. 2A-10 and 14A-14B, FIGS. 2A and 2B depict different side views of one embodiment of the anchoring device 12; FIGS. 3-6 depict side views of the device 12, according to various embodiments; FIGS. 7-9 depict top views of the device 12 (viewing the device 12 such that the proximal end is in the foreground and the distal end is in the background), according to various embodiments; and FIG. 10 depicts a bottom view of the device 12 (viewing the device 12 such that the distal end is in the foreground and the proximal end is in the background), according to one embodiment. In addition, FIG. 14A depicts a side view of another embodiment of the device 12, while FIG. 14B depicts a perspective view of that embodiment.

Each embodiment of the device 12 has an elongate body 22 with a substantially circular or oval-shaped cross-section that is made up of two body portions (also referred to as “elongate body portions” or “elongate body structures”) 22A, 22B. The cross-section of the body 22 of the device is defined by bands (also referred to herein as “ribs”) 24. In some embodiments, the bands 24 may comprise two sets of substantially semicircular bands 24A, 24B, wherein one set of bands 24A extends radially in a semi-circular fashion around the circumference of the first body portion 22A and a second set of bands 24B extends radially in a semi-circular fashion around the circumference of the second body portion 22B, while other configurations are possible.

In some embodiments, the first body portion 22A with the first set of semi-circular bands 24A defines one half of the lumen 18, while the corresponding second body portion 22B with the second set of semi-circular bands 24B defines the second, opposite half of the lumen 18.

In some embodiments, the bands 24A, 24B are attached to and supported by at least one elongate member or set of elongate members (also referred to as “spines” or “supports”) 26 that extend longitudinally along the length of the body 22.In some implementations, shown for example in FIGS. 2A-4, each body portion 22A, 22B has a single spine 26A, 26B.

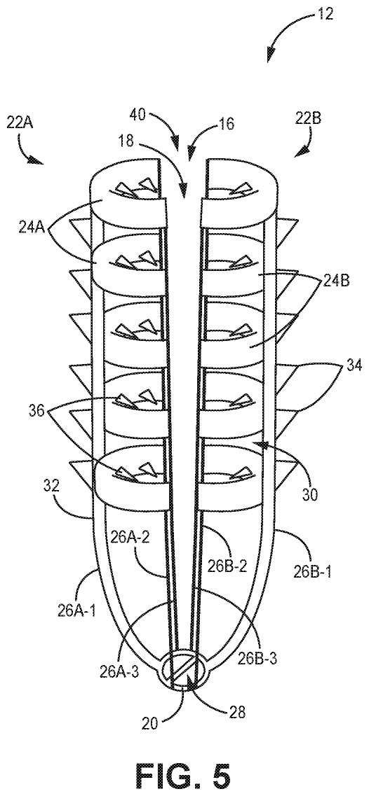

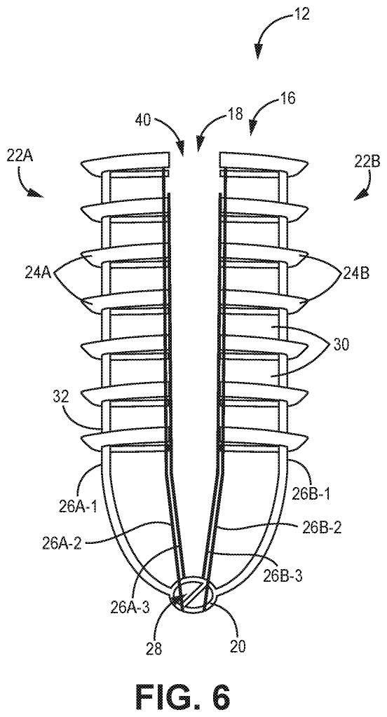

In the particular implementation shown in FIGS. 5 and 6, each body portion 22A, 22B has three spines 26. The first body portion 22A has a center spine 26A-1 and two lateral spines 26A-2-, 26A-3. The second body portion 22B has a center spine 26B-1, and two lateral spines 26B-2, 26B-3. Each spine 26 is configured to be attached to the bands 24A, 24B, respectively, of the device 12 to form the two body portions 22A, 22B.

In some embodiments, the spines 26 run substantially perpendicular to the bands 24 and parallel to the lumen 18 in a longitudinal fashion as mentioned above. In some embodiments, the spines 26 may also help to define the two semi-circular body portions 22A, 22B of the device 12.

The device 12 has an opening 16 defined at the proximal end that is in fluidic communication with an interior channel or lumen 18 defined by the semicircular bands 24A, 24B, as mentioned above. The lumen 18 is defined along a length of the elongate body 22 such that the lumen 18 is substantially parallel to the longitudinal axis of the body 22.

Further, the distal end of the body 22 has a distal connection structure 20. In various embodiments, the connection structure 20 defines an opening (also referred to as an “eyelet” or “grommet”) 28 such that the opening 28 is configured to allow a suture 14 (or, in certain embodiments, the tendon/graft 2 itself, as will be discussed in detail below) to be threaded therethrough, as described above. Exemplary openings 20 are depicted in FIGS. 2A, 2B, 5, 6, and 8-11B. In some embodiments, the connection structure 20 is a bar 20, such that suture 14 may be looped around the bar. Exemplary bars 20 are depicted in FIGS. 7, 14A, and 14B. The connection structure 20 is configured to connect first body portion 22A and the second body portion 22B. In some embodiments, the connection structure 20 is operatively engaged with the spines 26 of the body 22 to form a joint or hinge, such that the body portions 22A, 22B may move with respect to one another, as will be discussed further below. In some embodiments, the connection structure 20 is a hinge or hinge-like structure.

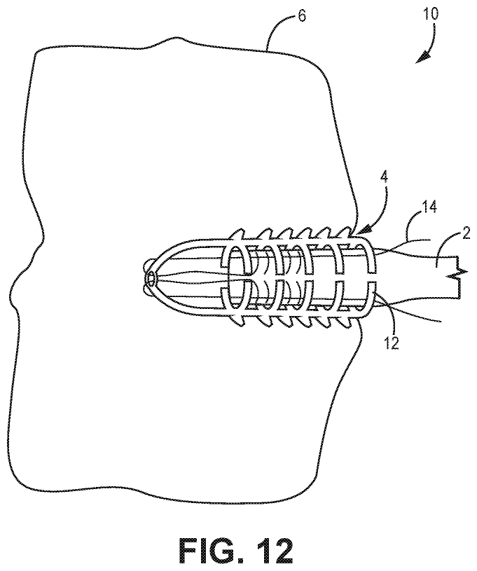

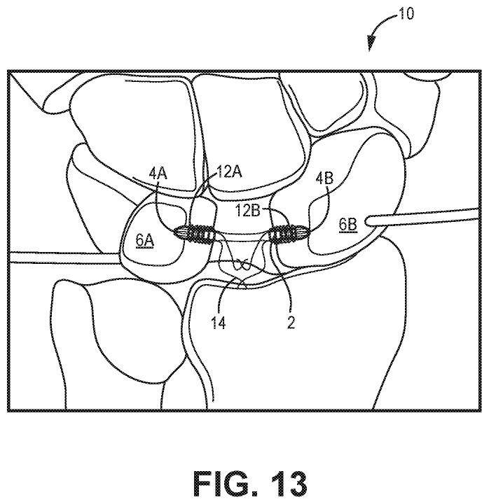

According to one embodiment, the body 19 of the device 12 is constructed such that there are a plurality of openings 30 defined within the body 22 of the device 12. For example, such openings 30 are identified in FIGS. 5, 6, 9, 14A, and 14B. Each of the openings 30 extends from the lumen 18 to the outer surface 32 of the body 22, thereby providing fluidic communication from the lumen 18 to the outer surface 32. These openings 30 are defined by the spaces or gaps between the bands 24A, 24B and the spines 26. The openings 30 are configured to create a tendon/bone interface when a tendon 2 is inserted into the device 12 and the device 12 is inserted into a bone cavity 8 (as best shown in FIGS. 12-13). Such a tendon/bone interface promotes biologic tendon bone healing.

In certain implementations, the body 22 has finger-like protrusions (also referred to as “teeth” or “barbs”) 34 disposed along the outer surface 32 of the body 22. Such protrusions 34 are depicted in FIGS. 3-5, 9, 14A, and 14B, for example. In some embodiments, the barbs 34 are placed along the exterior surface of the spines 26 and bands 24A, 24B and extend outwardly. According to some embodiments, the outer barbs 34 are disposed such that the protrusion (or sharp end) of each barb 34 extends proximally (toward the proximal end of the body 22). These “proximally pointing” outer barbs 34 are configured such that when the device 12 is inserted into a bore 4 (such as one of the bores 4, 4A, 4B of FIGS. 12 and 13), the barbs 34 become engaged with the inner wall of the bone bore 4 and prevent (or create resistance to) the device from being removed from the bone, thereby creating a stable anchor.

In an alternative embodiment, shown best in FIG. 6, the bands 24A, 24B are configured to be integral with finger-like protrusions 34. That is, in these implementations, the bands 24A, 24B are substantially triangular and disposed on the spines 26 such that a point of the triangular band 24A, 24B extends proximally, in a fashion similar to the barbs 34 described above. In various implementations, the triangular bands 24A, 24B engage with the inner wall of the bone bore 4 in a fashion similar to the barbs 34 to prevent the device 12 from being removed from the bone, creating a stable anchor. In some embodiments, the device 12 does not include distinct outer barbs 34.

In various embodiments, the device 12 has internal finger-like protrusions (also referred to as “teeth” or “barbs”) 36 that are disposed along the inner wall 38 of the lumen 18 such that the barbs 36 extend into the lumen 18, shown best, for example, in FIGS. 7-10, 14A, and 14B. In some embodiments, the barbs 36 are placed along the interior surface of the spines 26 and/or bands 24A, 24B. Opposite the outer barbs 34, the inner barbs 36, in certain implementations, are disposed such that the protrusion (or sharp end) of each barb 36 extends distally toward the distal end of the body 22. Thus, when a tendon 2 (such as tendon 2 in FIG. 1) is inserted into the lumen 18, the inner barbs 36 prevent the tendon from (or increase the resistance to the tendon) being removed from the device 12, thereby creating a stable anchor.

In various embodiments, the inner barbs 36 are blunted such as to grasp a tendon 2 upon compression, but not tear or rip the tendon when the tendon is pulled or stretched.

FIGS. 14A and 14B depict another embodiment of the anchoring device 12. In this implementation, the connection structure 20 of the anchoring device 12 is a connecting bar 20 as best shown in FIG. 14B. Further, in this implementation, the device 12 is configured to receive the tendon or tendon graft 2 such that the tendon/graft 2 is disposed within the anchoring device 12 such that the end of the tendon/graft 2 is threaded around the connecting bar 20 as best shown in FIG. 14B. In other words, the tendon/graft 2 is threaded around the bar 20 instead of a suture. The threading of the tendon/graft 2 can allow tensioning of the tendon without requiring a predetermined tendon/graft length. That is, the leading end of the tendon/graft 2 can be urged or pulled into the lumen 18 and around the connecting bar 20, and as the leading end of the tendon/graft 2 is urged out of the lumen 18 of the anchor 12, the desired amount of tension can be applied to the length of tendon/graft 2 between the device 12 as shown and the opposing device (not shown) at the other end of the tendon/graft 2 by pulling on the leading end of the tendon graft 2 (rather than a suture). Thus, this embodiment of the anchor 12 can simplify a reconstruction in that a predetermined tendon/graft length measurement is not required, since a longer tendon/graft can be used that need not be of a specific length.

Further, this exemplary anchoring device 12 implementation has both external barbs 34 disposed along the outer surface 32 and internal barbs 36 that are disposed along the inner wall 38. Thus, the external barbs 34 can function as described above, and the internal barbs 36 can function as as described above as well. Further, in certain embodiments, the internal barbs 36 are disposed such that some of the internal barbs 36A are oriented proximally to prevent the lead portion of the tendon/graft 2 from moving distally back toward and around the bar 20 and some of the internal barbs 36B are oriented distally to prevent the follow portion of the tendon/graft 2 from moving proximally toward and out of the opening 16, thereby preventing the tendon/graft 2 from being unthreaded and removed from the anchoring device 12. According to one exemplary implementation, as shown all of the barbs 36B on the first body portion 22A are oriented distally such that the length of tendon/graft 2 disposed adjacent to that body portion 22A is prevented from being urged proximally, while all of the barbs 36A on the second body portion 22B are oriented proximally such that the length of the tendon/graft 2 disposed adjacent to that body portion 22B is prevent from being urged distally. Thus, the barbs 36A, 36B are arranged to prevent removal/unthreading of the tendon/graft 2.

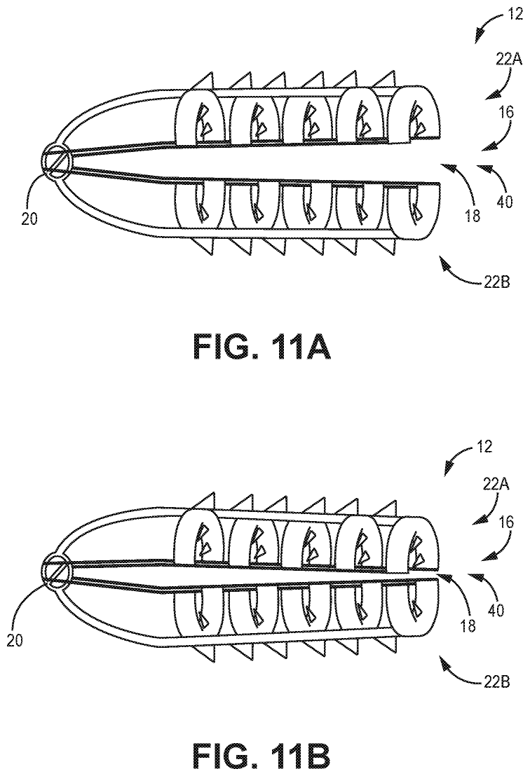

In certain embodiments, the device 12 may have elongate openings 40 along the body 22. Exemplary elongate openings 40 are depicted in FIGS. 2A, 4-11B, and 14A-14B. In some embodiments, the elongate openings 40 are defined by the two body portions 22A, 22B such that the openings 40 separate the two body portions 22A, 22B. These elongate openings 40 extend from the opening 16 to the connecting structure 20. The elongate openings 40, of some embodiments, are configured to allow for expansion and/or compression of the device 12. This expansion and compression of the device may exhibit itself by movement of the two body portions 22A, 22B farther apart or closer together in relation to each other, as will be further discussed in reference to FIGS. 11A and 11B.

Turning to FIGS. 11A and 11B, in certain embodiments as mentioned above, the device 12 can be configured to move between various positions (or configurations) such as a neutral position, an expanded position (as shown in FIG. 11A), and a compressed position (as shown in FIG. 11B). In the neutral (or non-expanded) position the opening 16 has a first diameter. In the expanded position (or configuration), the opening 16 is larger because the two body portions 22A, 22B are farther apart at the proximal end, such that the opening 16 has a second diameter larger than the first diameter when the device 12 is in a neutral configuration. This expanded position facilitates insertion of a tendon 2 into the lumen 18, as best shown in FIG. 11A. In the compressed position (or configuration), the opening 16 is smaller, defining a third diameter smaller than the second diameter when the device 12 is in an expanded configuration. In some embodiments, the third diameter in the compressed position is smaller than the first diameter when the device 12 is in a neutral position. This compressed configuration facilitates grasping of a tendon 2 within the lumen 18, as best shown in FIG. 11B.

As noted above, the distal end of the device 12 and connecting structure 20 is configured to act like a hinge or joint for purposes of the device 12 moving between the various positions described above, because the two body portions 22A, 22B are coupled to each other at that point. The elongate openings 40 along the device 12 facilitate the flexible, hinge like movement of the device 122.

As mentioned above, FIG. 11A depicts the device 12 in an expanded position, in accordance with one implementation. That is, the diameter of the opening 16 is larger by comparison to a non-expanded or neutral configuration. The device 12 may be urged into the expanded configuration by urging the two body portions 22A, 22B away from each other, thereby allowing for insertion of a tendon 2 with a diameter larger than the diameter of the neutral or non-expanded opening 16 of the device 12.

As also mentioned above, FIG. 11B depicts the device 12 in the compressed position, in accordance with one implementation. After a tendon is positioned within the lumen 18, the body 22 can be inserted into a bore, as shown in FIG. 12. In certain implementations, as the device 12 is urged into the bore 4, the outer surface 32 of the body 22 is urged toward the lumen 18, thereby causing the body 22 to be urged into the compressed configuration, discussed above. In the compressed configuration, the tendon 2 disposed within the lumen 18 is grasped by the body portion 22A, 22B, and the inner barbs 36, thereby attaching the device 12 to the tendon 2. This compression and grasping prevents the tendon 2 from being removed from the device 12.

FIGS. 12 and 13 depict the system 10 in use, and specifically in use within a patient's wrist, according to one embodiment. In one implementation, two anchoring devices 12A, 12B are inserted into the bores 4A, 4B in two bones 6A, 6B as shown to repair an injury, such as, for example, a ligament rupture. The two devices 12A, 12B are coupled to opposite ends of a tendon graft 2 (or, put another way, the tendon graft 2 is coupled at opposite ends to the devices 12A, 12B) for implantation, as discussed above, such that the insertion of the devices 12A, 12B into the bones 6A, 6B results in the tendon 2 being attached thereto.

The openings (also referred to as “cavities,” “drill holes,” or “bores”) 4A, 4B in the bones 6A, 6B may be created using a drill, awl, or other device or technique known to those of skill in the art. Each bore 4A, 4B may be smaller in diameter than the diameter of the anchoring devices 12A, 12B with the tendon 2 inserted therein. The anchoring devices 12A, 12B are inserted into the bores 4A, 4B, thereby compressing the devices 12A, 12B. As discussed above, such compression of each device 12A, 12B causes grasping of the tendon 2 and attachment of the devices 12A, 12B to the tendon 2, creating a stable anchor without crushing the tendon 2.

After insertion of the device 12 into bone 6, the suture 14 may continue to be pulled such that the tension on the tendon 2 may continue to be adjusted and increased. Additionally, the suture 14 ends may be tied together to augment the repair. That is, as is understood in known repair procedures without sutures, the repaired tendon has a tendency to stretch or elongate over time. In the embodiments herein, use of the sutures 14 can provide additional support to counter the tendon elongation. Thus, the additional support provided by the sutures 14 increases the strength of the repair and minimizes tendon creep.

Although the present invention has been described with reference to preferred embodiments, persons skilled in the art will recognize that changes may be made in form and detail without departing from the spirit and scope of the invention.

What is claimed is:

1. An anchoring device for repairing a ligament comprising:
(a) first and second elongate body portions, each elongate body portion comprising:
(i) at least one elongate member having a first end and a second end; and
(ii) a plurality of bands coupled to the at least one elongate member;
(b) a connection structure disposed at a distal end of the at least one elongate member connecting the first and second elongate body portions; and
(c) a lumen defined by the first and second elongate body portions,
wherein the first and second elongate body portions are movable in relation to each other via the connection structure.

2. The anchoring device of claim 1, wherein each of the first and second elongate body portions has a semi-circular cross-section.

3. The anchoring device of claim 1, wherein the lumen is configured to accept a tendon graft.

4. The anchoring device of claim 3, wherein the device is shaped for insertion into a bore.

5. The anchoring device of claim 4, wherein the first and second elongate body portions are configured to compress the tendon graft upon insertion into the bore.

6. The anchoring device of claim 1, wherein each of the plurality of bands further comprise a first side and a second side.

7. The anchoring device of claim 6, further comprising a plurality of barbs on the first side of each of the plurality of bands.

8. A method of repairing a ligament comprising:
attaching a suture to a first end of a tendon;
providing an anchoring device, wherein the anchoring device comprises:
(a) first and second elongate body portions, each of the first and second elongate body portions comprising:
(i) at least one elongate member having a proximal end and a distal end and
(ii) one or more bands coupled to the at least one elongate member;
(b) a connection disposed at a distal end of the at least one elongate member, the connection configured to allow movement of the first and second elongate body portion relative to each other; and
(c) a lumen defined by the first and second elongate body portions, the lumen having a first diameter;
creating a bore in a bone;
inserting the first end of the tendon into the lumen of the anchoring device; and
inserting the anchoring device into the bore.

9. The method of claim 8, further comprising urging the first and second elongate body portion toward each other such that the lumen is compressed to a second diameter, wherein the second diameter is smaller than the first diameter.

10. The method of claim 9, further comprising urging the first and second elongate body portions away from each other such that the lumen is expanded to a third diameter, wherein the third diameter is larger than the first diameter.

11. The method of claim 10, wherein the anchoring device further comprises a plurality of barbs disposed on the one or more bands and the plurality of barbs extending into the lumen.

12. The method of claim 8, further comprising threading the suture through the lumen.

13. The method of claim 8, further comprising tensioning the tendon.

14. An anchoring device comprising:
(a) an elongate body comprising:
(i) a first elongate body structure comprising:
(A) a first central support and
(B) a plurality of first ribs, wherein each of the plurality of first ribs is coupled to the first central support;
(ii) a second elongate body structure disposed adjacent to the first elongate body structure, the second elongate body structure comprising:
(A) a second central support and
(B) a plurality of second ribs, wherein each of the plurality of second ribs is coupled to the second central support;
(b) a lumen defined between the first and second elongate body structures, wherein the lumen has a substantially circular cross-section and a first diameter; and
(c) a distal connection structure disposed at a distal end of the elongate body, the distal connection structure coupled to the first and second central supports, wherein the first and second central supports are moveable in relation to each other via the distal connection structure.

15. The anchoring device of claim 14, wherein the distal connection structure is a hinge-like structure, wherein the first and second central supports are moveable between an expanded position and a non-expanded position at the hinge-like structure.

16. The anchoring device of claim 14, further comprising a plurality of protrusions extending outwardly from the first and second elongate body structures.

17. The anchoring device of claim 14, wherein the plurality of first ribs and the plurality of second ribs are substantially triangular such that a point extends outwardly form the first and second elongate body structures.

18. The anchoring device of claim 14, further comprising a plurality of barbs extending inwardly from the first and second elongate body structures.

19. The anchoring device of claim 14, wherein the distal connection structure is an eyelet.

20. The anchoring device of claim 14, wherein the distal connection structure is a bar.

21. The anchoring device of claim 14, further comprising:
(a) a plurality of protrusions extending outwardly from the first and second elongate body structures; and
(b) a plurality of barbs extending inwardly from the first and second elongate body structures.

22. The anchoring device of claim 21, wherein at least two of the plurality of barbs extending inwardly are oriented distally and at least two of the plurality of barbs extending inwardly are oriented proximally.

23. The anchoring device of claim 14, wherein the distal connection structure is sized and shaped to receive a suture or tendon such that the suture or tendon is disposed around or threaded through the distal connection structure.
